(19) United States
(12) Patent Application Publication
(10) Pub. No.: US 2003/0130355 A1
(43) Pub. Date: Jul. 10, 2003

(54) Therapeutic agents

(76) Inventors: David John Heal, Nottingham (GB); Sharon Crawford Cheetham, Nottingham (GB)

Correspondence Address: BROMBERG & SUNSTEIN LLP, 125 SUMMER STREET, BOSTON, MA, 02110-1618, US

(21) Appl. No.: 10/204,392

(22) PCT Filed: Feb. 20, 2001
(86) PCT No.: PCT/EP01/01894

(30) Foreign Application Priority Data
Feb. 22, 2000 (GB) 0004003.0

(51) Int. Cl. A61K 31/137

(52) U.S. Cl. 514/650

(57) ABSTRACT

The present invention provides a method of treating and preventing obesity and related co-morbid conditions comprising the administration of a therapeutically effective amount of one or more monoamine reuptake inhibitors which are serotonin reuptake inhibitors and/or noradrenaline reuptake inhibitors and a 5-HT_{1A} agonist to a patient in need thereof.

This invention relates to a method for treating and preventing obesity and related co-morbid conditions associated with obesity and to products and pharmaceutical compositions suitable for use in such a method. More particularly, the invention relates to a method for the treatment of obesity and related co-morbid conditions associated with obesity comprising the administration of a 5-HT_{1A} agonist in combination with a monoamine reuptake inhibitor which is a serotonin (5-HT) reuptake inhibitor and/or a noradrenaline reuptake inhibitor.

Several monoamine reuptake inhibitors, for example sibutramine, are useful in the treatment of obesity. However, some of these compounds have undesirable cardiovascular side-effects, for example increased heart-rate and increased blood pressure. It has now surprisingly been found that combining a monoamine reuptake inhibitor which is a serotonin reuptake inhibitor and/or a noradrenaline reuptake inhibitor with a 5-HT_{1A} agonist reduces the cardiovascular side-effects without diminishing the effect of the compounds in treating obesity and related co-morbid conditions.

The present invention provides a method of treating and preventing obesity and related co-morbid conditions comprising the administration of a therapeutically effective amount of one or more monoamine reuptake inhibitors which are serotonin reuptake inhibitors and/or noradrenaline reuptake inhibitors and a 5-HT_{1A} agonist to a patient in need thereof. Alternatively the present invention provides one or more monoamine reuptake inhibitors, which are serotonin reuptake inhibitors and/or noradrenaline reuptake inhibitors, and one or more 5-HT_{1A} agonists for use in the treatment of obesity.

The monoamine reuptake inhibitor and the 5-HT_{1A} agonist may be administered simultaneously, separately or sequentially. Therefore, the monoamine reuptake inhibitor and the 5-HT_{1A} agonist may be provided as a single formulation (for simultaneous administration) or as a kit of parts (for separate or sequential administration).

In one preferred embodiment the monoamine reuptake inhibitor is a selective noradrenaline reuptake inhibitor. In a second preferred embodiment the monoamine reuptake inhibitor is a serotonin and noradrenaline reuptake inhibitor.

In another aspect the present invention provides a method of treating and preventing obesity and related co-morbid conditions comprising the administration of a therapeutically effective amount of a serotonin and noradrenaline reuptake inhibitor and a 5-HT_{1A} agonist to a patient in need thereof.

In yet another aspect the present invention provides a method of reducing the cardiovascular side-effects associated with treating and preventing obesity and related co-morbid conditions comprising the administration of a therapeutically effective amount of a serotonin and noradrenaline reuptake inhibitor and a 5-HT_{1A} agonist to a patient in need thereof.

In yet another aspect the present invention also provides a method for treating obesity and related co-morbid conditions comprising the administration of a noradrenaline reuptake inhibitor and a 5-HT_{1A} agonist to a patient in need thereof.

In yet another aspect the present invention provides a method of reducing the cardiovascular side-effects associated with treating and preventing obesity and related co-morbid conditions comprising the administration of a therapeutically effective amount of a noradrenaline reuptake inhibitor and a 5-HT_{1A} agonist to a patient in need thereof.

The present invention is surprising because it is known that the 5-HT_{1A} receptor agonist 8-OH-DPAT increases food intake in rats (Dourish et al, Psychopharmacology 1985, 86, 197-204).

Suitable serotonin and/or noradrenaline reuptake inhibitors include but are not limited to sibutramine or an enantiomer thereof or a sibutramine metabolite or an enantiomer thereof as described in Formula I below, reboxetine, desipramine, Org 4428, nisoxetine, venlafaxine, amitriptyline, milnacipran, duloxetine, tomoxetine and LY368975 (thionisoxetine), including pharmaceutically acceptable salts thereof and individual enantiomers, racemates or other mixtures of enantiomers.

A preferred serotonin and/or noradrenaline reuptake inhibitor is N-{1-[1-(4-chlorophenyl)cyclobutyl]-3-methylbutyl}-N,N-dimethylamine or a salt thereof, for example the hydrochloride salt, known as sibutramine hydrochloride. A preferred form of this hydrochloride is its monohydrate, known as sibutramine hydrochloride monohydrate.

Suitable 5-HT_{1A} agonists include but are not limited to buspirone, gepirone, pindolol, flesinoxan, ipsapirone, A-74283, RU-24969, 8-OH-DPAT, FG 5938, S20499 (alnespirone), BAYx3702, tandospirone, SUN8399 and LY228729, including pharmaceutically acceptable salts thereof and individual enantiomers, racemates or other mixtures of enantiomers.

In another aspect the present invention provides a serotonin and noradrenaline reuptake inhibitor of Formula I

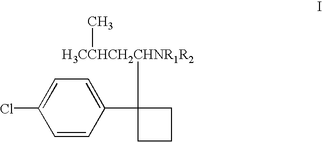

including enantiomers and pharmaceutically acceptable salts thereof, in which R₁ and R₂ are independently H or methyl, and a 5-HT_{1A} agonist for simultaneous, separate or sequential use for the prevention and/or treatment of obesity and co-morbid conditions associated with obesity.

In yet another aspect the present invention provides a compound of Formula I including enantiomers and pharmaceutically acceptable salts thereof, in which R₁ and R₂ are independently H or methyl, and a 5-HT_{1A} agonist as a combined preparation for use in the prevention and/or treatment of obesity and co-morbid conditions associated with obesity. The combined preparation may be a composition or a kit of parts.

In a further aspect the present invention provides a product containing a compound of Formula I including enantiomers and pharmaceutically acceptable salts thereof, in which R₁ and R₂ are independently H or methyl, and a 5-HT_{1A} agonist as a combined preparation for use in the prevention and/or treatment of obesity and co-morbid conditions associated with obesity.

In yet another aspect the present invention provides the use of a compound of Formula I including enantiomers and pharmaceutically acceptable salts thereof, in which R₁ and R₂ are independently H or methyl, in the manufacture of a medicament for the prevention and/or treatment of obesity and co-morbid conditions associated with obesity in a patient who is also receiving treatment with a 5-HT_{1A} receptor agonist.

In a further aspect, the present invention provides a method of treating obesity and co-morbid conditions associated with obesity comprising the administration of an adjunctive therapy comprising a therapeutically effective amount of a compound of Formula I and a 5-HT_{1A} receptor agonist to a patient in need thereof.

The invention also provides the use of the above combination of drugs in the manufacture of a medicament for the treatment of obesity and co-morbid conditions associated with obesity. Additionally, it provides the combination for use in the treatment of obesity and co-morbid conditions associated with obesity.

The invention further provides the use of a monoamine reuptake inhibitor which is a serotonin reuptake inhibitor and/or a noradrenaline reuptake inhibitor and a 5-HT_{1A} agonist in the manufacture of a medicament for the treatment of obesity and co-morbid conditions associated with obesity by simultaneous, separate or sequential administration of the monoamine reuptake inhibitor and the 5-HT_{1A} agonist.

Compounds of Formula I may exist as salts with pharmaceutically acceptable acids. The present invention includes all such salts. Examples of such salts include hydrochlorides, hydrobromides, sulphates, methanesulphonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid.

Certain compounds of Formula I and their salts may exist in more than one crystal form and the present invention includes each crystal form and mixtures thereof. Certain compounds of Formula I and their salts may also exist in the form of solvates, for example hydrates, and the present invention includes each solvate and mixtures thereof.

Compounds of Formula I contain a chiral centre, and exist in different optically active forms. These compounds exist in two enantiomeric forms and the present invention includes both enantiomers and mixtures of enantiomers. The enantiomers may be resolved by methods known to those skilled in the art, for example by formation of diastereoisomeric salts which may be separated, for example, by crystallisation; formation of diastereoisomeric derivatives or complexes which may be separated, for example, by crystallisation, gas-liquid or liquid chromatography or gas-liquid or liquid chromatography in a chiral environment, for example on a chiral support, for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesised by asymmetric synthesis using optically active reagents, substrates, catalysts or solvents, or by converting one enantiomer into the other by asymmetric transformation.

The preparation and use of compounds of Formula I, such as N-{1-[1-(4-chlorophenyl)cyclobutyl]-3-methylbutyl}-N,N-dimethylamine and salts thereof, in the treatment of depression is described in British Patent Specification 2098602. The use of compounds of Formula I such as N-{1-[1-(4-chlorophenyl)cyclobutyl]-3-methylbutyl}-N,N-dimethylamine and salts thereof in the treatment of Parkinson's disease is described in published PCT application WO 88/06444. The use of N-{1-[1-(4-chlorophenyl)cyclobutyl]-3-methylbutyl}-N,N-dimethylamine and salts thereof in the treatment of cerebral function disorders is described in U.S. Pat. No. 4,939,175. The use of N{1-[1-(4-chlorophenyl)cyclobutyl]-3-methylbutyl}-N,N-dimethylamine hydrochloride in the treatment of obesity is described in European Patent Number 397831. A particularly preferred form of this compound is N-{1-[1-(4-chlorophenyl)cyclobutyl]-3-methylbutyl}-N,N-dimethylamine hydrochloride monohydrate (sibutramine hydrochloride monohydrate) which is described in European Patent Number 230742. The use of N-{1-[1-(4-chlorophenyl)cyclobutyl]-3methylbutyl}-N,N-dimethylamine and salts thereof for improving the glucose tolerance of humans having Impaired Glucose Tolerance or Non-insulin Dependent Diabetes Mellitus is described in published PCT application WO95/20949.

Preferred compounds of Formula I are:

N-{1-[1-(4-chlorophenyl)cyclobutyl]-3-methylbutyl}-N,N-dimethylamine,

N-{1-[1-(4-chlorophenyl)cyclobutyl]-3-methylbutyl}N-methylamine, and

1-[1-(4-chlorophenyl)cyclobutyl]-3-methylbutylamine

including racemates, individual enantiomers and mixtures thereof, and pharmaceutically acceptable salts thereof.

Specific enantiomers of Formula I are:

(R)-(+)-N-{1-[1-(4-chlorophenyl)cyclobutyl]-3-methylbutyl}-N,N-dimethylamine,

(S)-(−)-N-{1-[1-(4-chlorophenyl)cyclobutyl]-3-methylbutyl}-N,N-dimethylamine,

(R)-(+)-N-{1-[1-(4-chlorophenyl)cyclobutyl]-3-methylbutyl}-N-methylamine,

(S)-(−)-N-{1-[1-(4-chlorophenyl)cyclobutyl]-3-methylbutyl}-N-methylamine,

(R)-(+)-1-[1-(4-chlorophenyl)cyclobutyl]-3-methylbutylamine, and

(S)-(−)-1-[1-(4-chlorophenyl)cyclobutyl]-3-methylbutylamine.

including pharmaceutically acceptable salts thereof.

A particularly preferred combination is a compound of Formula I, especially sibutramine, and flesinoxan. Flesinoxan is described in EP138,280 and U.S. Pat. No. 4,833,142

The term “co-morbid conditions associated with obesity” as used in this document means medical conditions known to those skilled in the art to be associated with obesity. The term includes but is not limited to the following: diabetes including non-insulin dependent diabetes mellitus, impaired glucose tolerance, hypertension, coronary thrombosis, stroke, eating disorders such as bulimia, anorexia, snacking and binge eating, lipid syndromes, hyperglycaemia and hyperlipidaemia in mammals particularly humans.

In addition the present invention may be useful in the treatment or prevention of metabolic diseases and conditions arising therefrom, for example non-exercise reduced or increased metabolic rate, sleep apnoea, weight gain associated with drug treatment, osteoarthritis, rheumatoid arthritis, gout, cancers associated with weight gain, menstrual dysfunction, gallstones, orthostatic hypertension and pulmonary hypertension.

The present invention may be useful in preventing cardiovascular disease, in aiding weight loss after pregnancy, reducing the craving to smoke and in aiding weight loss after smoking cessation. The present invention may also be useful in lowering uric acid levels and lipid levels in mammals, particularly humans.

In the method of the present invention the monoamine reuptake inhibitor and the 5-HT_{1A} agonist may be administered concomitantly or concurrently, for example in the form of separate dosage units to be used simultaneously, separately or sequentially.

The amount of each compound to be administered will depend on a number of factors including the age of the patient, the severity of the condition and the past medical history of the patient and always lies within the sound discretion of the administering physician but it is generally envisaged that the dosage of the monoamine reuptake inhibitor to be administered will be in the range 0.1 to 1000 mg preferably 1 to 30 mg per day given in one or more doses and more preferably 5 mg, 10 mg, 15 mg, 20 mg, 25 mg or 30 mg per day and most preferably 10 mg, 15 mg or 20 mg. The dosage of the 5-HT_{1A} agonist to be administered will be in the range of 0.1 to 1500 mg given in one or more doses, preferably three times daily, more preferably in the range of 5 to 500 mg and most preferably in the range of 10 to 250 mg. The compound of Formula I, preferably sibutramine hydrochloride monohydrate, may be administered in any of the known pharmaceutical dosage forms. The compounds are preferably administered orally.

In a preferred aspect of the present invention sibutramine hydrochloride monohydrate is administered once daily, preferably first thing in the morning, and a 5-HT_{1A} agonist is administered once, twice or thrice daily. Preferably the dose of sibutramine hydrochloride monohydrate is 5 mg, 10 mg, 15 mg, 20 mg, 25 mg or 30 mg administered once daily and the dose of 5-HT_{1A} agonist is in the range of 1 to 250 mg administered three times daily either with or before meals. Most preferably the dose of sibutramine hydrochloride monohydrate is given prior to the first dose of 5-HT_{1A} agonist, preferably in the range of 30 minutes to 3 hours, for example 30 minutes, 1 hour, 1.5 hours, 2 hours, 2.5 hours or 3 hours, before the first dose of 5-HT_{1A} agonist.

In another aspect of the present invention there is provided a pharmaceutical composition comprising a compound of Formula IEINBETTEN including enantiomers and pharmaceutically acceptable salts thereof, in which R₁ and R₂ are independently H or methyl, and a 5-HT_{1A} agonist in conjunction with a pharmaceutically acceptable diluent or carrier.

Oral dosage forms are the preferred compositions for use in the present invention and these are the known pharmaceutical forms for such administration, for example tablets, capsules, granules, syrups and aqueous or oil suspensions. The excipients used in the preparation of these compositions are the excipients known in the pharmacist's art. Tablets may be prepared from a mixture of the active compounds with fillers, for example calcium phosphate; disintegrating agents, for example maize starch; lubricating agents, for example magnesium stearate; binders, for example microcrystalline cellulose or polyvinylpyrrolidone and other optional ingredients known in the art to permit tableting the mixture by known methods. The tablets may, if desired, be coated using known methods and excipients which may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. The tablets and capsules may conveniently each contain 1 to 300 mg of the monoamine reuptake inhibitor compound, particularly 1 to 30 mg of a compound of Formula I, and 1 to 500 mg of a 5-HT_{1A} agonist.

Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (for example, water) before ingestion. The granules may contain disintegrants, eg an effervescent couple formed from an acid and a carbonate or bicarbonate salt to facilitate dispersion in the liquid medium.

The compounds of Formula I and/or a 5-HT_{1A} receptor agonist may be formulated into a composition which the patient retains in his mouth so that the active compounds are administered through the mucosa of the mouth.

Dosage forms of the compounds of Formula I and/or a 5-HT_{1A} receptor agonist suitable for rectal administration are the known pharmaceutical forms for such administration, for example, suppositories with cocoa butter or polyethylene glycol bases.

Dosage forms of the compounds of Formula I and/or a 5-HT_{1A} receptor agonist suitable for parenteral administration are the known pharmaceutical forms for such administration, for example sterile suspensions or sterile solutions in a suitable solvent.

Dosage forms of the compounds of Formula I and/or a 5-HT_{1A} receptor agonist for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as a mineral oil, petrolatum and/or a wax, e.g. paraffin wax or beeswax, together with a potential transdermal accelerant such as dimethyl sulphoxide or propylene glycol. Alternatively the active compounds may be dispersed in a pharmaceutically acceptable cream, gel or ointment base. The amount of each active compound contained in a topical formulation should be such that a therapeutically effective amount of each compound is delivered during the period of time for which the topical formulation is intended to be on the skin.

The compounds of Formula I and/or a 5-HT_{1A} receptor agonist may be formulated into a composition which is dispersed as an aerosol into the patient's oral or nasal cavity. Such aerosols may be administered from a pump pack or from a pressurised pack containing a volatile propellant.

The compound of Formula I and/or a 5-HT_{1A} receptor agonist may also be administered by continuous infusion either from an external source, for example by intravenous infusion or from a source of the compound placed within the body. Internal sources include implanted reservoirs containing the compounds to be infused which is continuously released for example by osmosis and implants which may be (a) liquid such as an oily suspension of the compounds to be infused for example in the form of a very sparingly water-soluble derivative such as a dodecanoate salt or a lipophilic ester or (b) solid in the form of an implanted support, for example of a synthetic resin or waxy material, for the compounds to be infused. The support may be a single body containing all the compounds or a series of several bodies each containing part of the compounds to be delivered. The amount of active compounds present in an internal source should be such that a therapeutically effective amount of each compound is delivered over a long period of time.

In some formulations it may be beneficial to use the compounds of the present invention in the form of particles of very small size, for example as obtained by fluid energy milling.

In the compositions of the present invention the active compounds may, if desired, be associated with other compatible pharmacologically active ingredients. Optionally vitamin supplements may be administered with the compounds of the present invention.

Pharmaceutical compositions incorporating both a compound of Formula I and a 5-HT_{1A} receptor agonist are important embodiments of the present invention. Such pharmaceutical compositions contain a therapeutically effective amount of each of the compounds. Each dosage unit may contain the daily doses of both compounds, or may contain a fraction of the daily dose, such as one-third of the doses. Alternatively, each dosage unit may contain the entire dose of one of the compounds, and a fraction of the dose of the other compound. In such case, the patient would daily take one of the combination dosage units, and one or more units containing only the other compound.

The use of compounds of the present invention in the manufacture of pharmaceutical compositions is illustrated by the following description. In this description the term “active compound” denotes either or both compounds of the invention unless otherwise stated.

a) Capsules

In the preparation of capsules, 10 parts by weight of active compound and 240 parts by weight of lactose are de-aggregated and blended. The mixture is filled into hard gelatin capsules, each capsule containing a unit dose or part of a unit dose of active compound.

b) Tablets

Tablets are prepared from the following ingredients.

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch are de-aggregated, blended and the resulting mixture is granulated with a solution of the polyvinyl-pyrrolidone in ethanol. The dry granulate is blended with the magnesium stearate and the rest of the starch. The mixture is then compressed in a tabletting machine to give tablets each containing a unit dose or a part of a unit dose of active compound.

Enteric Coated Tablets

Tablets are prepared by the method described in (b) above. The tablets are enteric coated in a conventional manner using a solution of 20% cellulose acetate phthalate and 3% diethyl phthalate in ethanol:dichloromethane (1:1).

d) Suppositories (Compound of Formula I Only)

In the preparation of suppositories, 100 parts by weight of active compound is incorporated in 1300 parts by weight of triglyceride suppository base and the mixture formed into suppositories each containing a therapeutically effective amount of active ingredient.

The invention is illustrated by the following Examples which are given by way of example only. The final product of each of these Examples was characterised by one or more of the following procedures: gas-liquid chromatography; high performance liquid chromatography, mass spectrometry, polarimetry, elemental analysis, x-ray crystallography, nuclear magnetic resonance spectroscopy and infrared spectroscopy.

The invention is also illustrated, by way of example only, in the accompanying drawings in which:

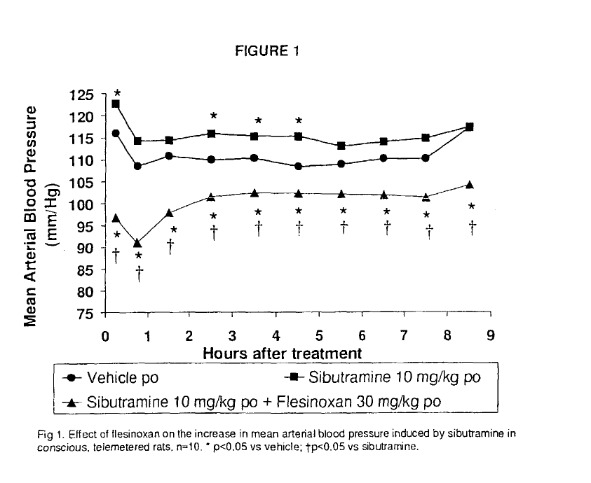

FIG. 1 is a plot of mean arterial blood pressure (mm/Hg) against time (hours) showing the effect of flesinoxan on the increase in arterial blood pressure induced by sibutramine in conscious, telemetered rats; and

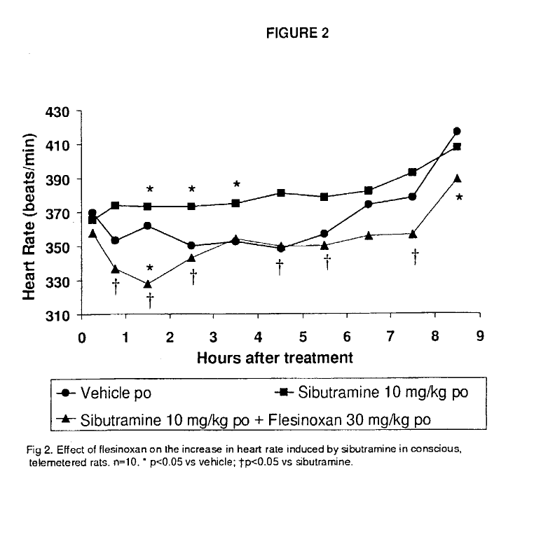

FIG. 2 is a plot of heart rate (beats/min) against time (hours) showing the effect of flesinoxan on the increase in heart rate induced by sibutramine in conscious, telemetered rats.

### EXAMPLES

### Example 1

The beneficial properties of especially preferred compounds of the present invention in reducing cardiovascular side-effects have been demonstrated in rat telemetry studies in which heart rate and blood pressure are recorded continuously over time. The methods used were similar to those described in:

Brockway, B P, Mills, P A & Azar, S H (1991) A new method for continuous chronic measurement of blood pressure, heart rate and activity in the rat via radio-telemetry. Clinical and Experimental Hypertension—Theory and Practice A13(5), 885-895 and

Guiol, C, Ledoussal, C & Surgé, J-M (1992) A radiotelemetry system for chronic measurement of blood pressure and heart rate in the unrestrained rat. Validation of method. Journal of Pharmacological and Toxicological Methods 28, 99-105.

In brief, male rats (n=10) were implanted with a telemetry device for the measurement of arterial blood pressure and heart rate. Two weeks following implantation each animal received a single oral dose of vehicle, one dose of sibutramine or a combination of sibutramine and flesinoxan in a cross-over design. Measurements of blood pressure and heart rate were taken from each rat in sequence every three minutes for nine hours following drug administration and averaged into appropriate time-bins. Statistical comparisons were by the least significant difference or multiple t-test.

In FIGS. 1 and 2, shaded circles relate to the administration of vehicle alone p.o., shaded squares relate to the administration of sibutramine at 10 mg/kg p.o. and shaded triangles relate to the administration of sibutramine at 10 mg/kg p.o. together with flesinoxan at 30 mg/kg p.o. Statistical comparisons made by the least significant difference or multiple t-test are shown as *=p<0.05 versus vehicle and t=p<0.05 versus sibutramine.

The 5-HT and noradrenaline reuptake inhibitor, sibutramine (10 mg/kg p.o.), produced a significant increase in mean arterial blood pressure in the conscious, telemetered rat model as shown in FIG. 1. This effect was completely reversed by co-administration of flesinoxan (30 mg/kg p.o.). Thus, the blood pressure of rats given both sibutramine and flesinoxan was significantly lower than that of animals given sibutramine alone and also the vehicle-treated control group throughout the nine hours following drug administration. Flesinoxan also prevented the increase in heart rate produced by sibutramine in the conscious, telemetered rats as shown in FIG. 2. The heart rates of rats given sibutramine and the 5-HT_{1A} agonist were significantly lower than those of animals given sibutramine alone and either similar to or lower than those of the control group.

### Example 2

The (+)-enantiomer of sibutramine was obtained by N,N-dimethylation of the (+)-enantiomer of the primary amine precursor which was obtained by resolution of racemic 1-[1-(4-chlorophenyl)cyclobutyl]-3-methylbutylamine, which was prepared according to GB 2098602. (S)-(+)-N-carbamoylphenylalanine was mixed with the racemic primary amine in methanol to give a diastereoisomeric mixture of salts. One salt crystallised preferentially from the mixture and this was recrystallised from methanol and basified to afford the (+)-primary amine, (R)-(+)-1-[1-(4-chlorophenyl)-cyclobutyl]-3-methylbutylamine, which had an optical purity >98% (by nmr).

Concentration of the filtrate gave the other salt which upon similar treatment gave the (−)-primary amine, (S)-(−)-1-[1-(4-chlorophenyl)cyclobutyl]-3-methyl-butylamine.

The two primary amines were N,N-dimethylated by methods described in GB 2098602 to give the tertiary amines, which were converted into their hydrochloride salts, (R)-(+)-N-{1-[1-(4-chlorophenyl)cyclobutyl]-3-methylbutyl}-N,N-dimethylamine hydrochloride and (S)-(−)-N-{1-[1-(4-chlorophenyl)cyclobutyl]-3-methylbutyl}-N,N-dimethylamine hydrochloride. Both the (+)-tertiary amine hydrochloride and the (−)-tertiary amine hydrochloride were at least 98% optically pure by nmr. Analytical data for these two samples are given below.

The two primary amines may also be monomethylated by methods described in GB 2098602 to give (R)-(+)-N-{1-[1-(4-chlorophenyl)cyclobutyl]-3-methylbutyl}-N-methylamine and (S)-(−)-N-{1-[1-(4-chlorophenyl)cyclobutyl]-3-methylbutyl}-N-methylamine.

Physicochemical Data for (R)-(+)-N-{1-[1-(4-chlorophenyl)cyclobutyl]-3-methylbutyl}-N,N-dimethylamine hydrochloride

m.p. 232° C.

[α]_{D}=+4.5° (c=2.61; ethanol)

Optical purity: >98% by nmr

Elemental analysis:

C₁₇H₂₆ClN. HCl requires: C=64.6; H=8.5; N=4.4; Cl=22.5%

Found: C=65.0; H=8.6; N 4.5; Cl=22.8%

The absolute stereochemistry was determined by X-ray crystallography and found to be R.

Physicochemical Data for (S)-(−)-N-{1-[1-(4-chlorophenyl)cyclobutyl]-3-methylbutyl}-N,N-dimethylamine Hydrochloride

m.p. 232° C.

[α]_{D}=−4.9° (C=3.8; ethanol)

Optical purity: >98% by nmr

Elemental analysis:

C₁₇H₂₆ClN. HCl requires: C=64.6; H=8.5; N=4.4; Cl=22.5%

Found: C=64.2; H=8.6; N=4.5; Cl=22.9%

The absolute stereochemistry was determined by X-ray crystallography and found to be S.

1. A method of treating and preventing obesity and related co-morbid conditions comprising the administration of a therapeutically effective amount of a monoamine reuptake inhibitor which is a serotonin reuptake inhibitor and/or a noradrenaline reuptake inhibitor and 5-HT_{1A} agonist to a patient in need thereof.

2. A composition or kit of parts comprising a monoamine reuptake inhibitor which is a serotonin reuptake inhibitor and/or a noradrenaline reuptake inhibitor and a 5-HT_{1A} agonist for use in the treatment of obesity and co-morbid conditions associated with obesity.

3. A method according to claim 1 wherein the serotonin and/or noradrenaline reuptake inhibitor is sibutramine or an enantiomer thereof or a sibutramine metabolite or an enantiomer thereof as described in Formula I 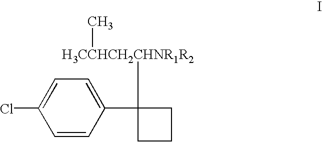
including enantiomers and pharmaceutically acceptable salts thereof, in which R₁ and R₂ are independently H or methyl, or reboxetine, desipramine, Org 4428, nisoxetine, venlafaxine, amitriptyline, milnacipran, duloxetine, tomoxetine and LY368975 (thionisoxetine), including pharmaceutically-acceptable salts thereof and individual enantiomers, racemates or other mixtures of enantiomers.

4. A method according to claim 3 wherein the serotonin and noradrenaline reuptake inhibitor is N-{1-[1-(4-chlorophenyl)cyclobutyl]-3-methylbutyl}-N,N-dimethylamine or a salt thereof.

5. A method according to claim 3 wherein the serotonin and noradrenaline reuptake inhibitor is (R)-(+)-N-{1-[1-(4-chlorophenyl)cyclobutyl]-3-methylbutyl}-N,N-dimethylamine or a salt thereof or (S)-(−)-N-{1-[1-(4-chlorophenyl)cyclobutyl]-3-methylbutyl}-N,N-dimethylamine or a salt thereof.

6. A method according to claim 3 wherein the serotonin and noradrenaline reuptake inhibitor is N-{1-[1-(4-chlorophenyl)cyclobutyl]-3-methylbutyl}-N-methylamine or a salt thereof, (R)-(+)-N-{1-[1-(4-chlorophenyl)cyclobutyl]-3-methylbutyl}-N-methylamine or a salt thereof or (S)-(−)-N-{1-[1-(4 chlorophenyl)-cyclobutyl]-3-methylbutyl}-N-methylamine or a salt thereof.

7. A method according to claim 3 wherein the serotonin and noradrenaline reuptake inhibitor is 1-[1-(4-chlorophenyl)cyclobutyl]-3-methylbutylamine or a salt thereof, (R)-(+)-1-[1-(4-chlorophenyl)cyclobutyl]-3-methylbutylamine or a salt thereof or (S)-(−)-1-[1-(4-chlorophenyl)cyclobutyl]-3-methylbutylamine or a salt thereof.

8. A method according to claim 4 wherein the serotonin and noradrenaline reuptake inhibitor is N-{1-[1-(4-chlorophenyl)cyclobutyl]-3-methylbutyl}-N,N-dimethylamine hydrochloride monohydrate.

9. A method according to any previous claim wherein the 5-HT_{1A} agonist is buspirone, gepirone, pindolol, flesinoxan, ipsapirone, A-74283, RU-24969, 8-OH-DPAT, FG 5938, S20499 (alnespirone), BAYx3702, tandospirone, SUN8399 and LY228729, including pharmaceutically acceptable salts thereof and individual enantiomers, racemates or other mixtures of enantiomers.

10. A method according to any one of claims 4, 5 or 8 wherein the serotonin and noradrenaline reuptake inhibitor is N-{1-[1-(4-chlorophenyl)cyclobutyl]-3-methylbutyl}-N,N-dimethylamine or a salt thereof and the 5-HT_{1A} agonist is flesinoxan.

11. A composition or kit of parts comprising a serotonin and noradrenaline reuptake inhibitor of Formula I as defined in claim 3 including enantiomers and pharmaceutically acceptable salts thereof, in which R₁ and R₂ are independently H or methyl, and a 5-HT_{1A} agonist for simultaneous, separate or sequential use for the prevention and/or treatment of obesity and co-morbid conditions associated with obesity.

12. A composition comprising a compound of Formula I as defined in claim 3 including enantiomers and pharmaceutically acceptable salts thereof, in which R₁ and R₂ are independently H or methyl, and a 5-HT_{1A} agonist as a combined preparation for use in the prevention and/or treatment of obesity and co-morbid conditions associated with obesity.

13. A kit of parts comprising a compound of Formula I as defined in claim 3 including enantiomers and pharmaceutically acceptable salts thereof, in which R₁ and R₂ are independently H or methyl, and a 5-HT_{1A} agonist for simultaneous, separate or sequential use in the prevention and/or treatment of obesity and co-morbid conditions associated with obesity.

14. Use of a compound of Formula I as defined in claim 3 including enantiomers and pharmaceutically acceptable salts thereof, in which R₁ and R₂ are independently H or methyl, in the manufacture of a medicament for the prevention and/or treatment of obesity and co-morbid conditions associated with obesity in a patient who is also receiving treatment with a 5-HT_{1A} receptor agonist.

15. Use of a monoamine reuptake inhibitor which is a serotonin reuptake inhibitor and/or a noradrenaline reuptake inhibitor and a 5-HT_{1A} agonist in the manufacture of a medicament for the treatment of obesity and co-morbid conditions associated with obesity by simultaneous, separate or sequential administration of the monoamine reuptake inhibitor and the 5-HT_{1A} agonist.
